(19) United States
(12) Patent Application Publication
(10) Pub. No.: US 2008/0234381 A1
(43) Pub. Date: Sep. 25, 2008

(54) Novel Trifluoromethoxy-Substituted Aryl Anilides

(75) Inventors: Preben Houlberg Olesen, Copenhagen NV (DK); Holger Claus Hansen, Vaerlose (DK); Lise Brown Christiansen, Lyngby (DK); Anders Klarskov Petersen, Naerum (DK); Flemming Elmelund Nielsen, Virum (DK); Birgit Sehested Hansen, Stenlose (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/914,629

(22) PCT Filed: May 23, 2006
(86) PCT No.: PCT/EP2006/062527

§ 371 Date: Mar. 24, 2008

(30) Foreign Application Priority Data
May 23, 2005 (EP) 05104342.0

(51) Int. Cl. A61K 31/192; C07C 229/02; A61P 3/04; A61P 9/10

(52) U.S. Cl. 514/577; 562/456

(57) ABSTRACT

The present invention relates to novel compounds that act as chemical uncouplers. Compounds of the invention are useful in the treatment, including prevention, of obesity, diabetes and a number of diseases or disorders associated therewith.

### FIELD OF THE INVENTION

The invention relates to novel trifluoromethoxy-substituted aryl anilides which are particularly interesting as chemical uncouplers, and which are useful, inter alia, in the treatment of obesity as well as diseases or disorders associated therewith.

### BACKGROUND OF THE INVENTION

Obesity is a well-known risk factor for the development of many very common diseases or disorders such as atherosclerosis, hypertension, type 2 diabetes (non-insulin dependent diabetes mellitus (NIDDM)), dyslipidemia, coronary heart disease, and osteoarthritis and various malignancies. It also causes considerable problems through reduced motility and decreased quality of life. The incidence of obesity in humans, and thereby also the incidence of diabetes-associated diseases or disorders is increasing throughout the entire industrialised world.

The term obesity implies an excess of adipose tissue. In this context obesity is best viewed as any degree of excess adiposity that imparts a health risk. The cut off between normal and obese individuals can only be approximated, but the health risk imparted by obesity is probably a continuum with increasing adiposity. In the context of the present invention, individuals with a body mass index (BMI=body weight in kilograms divided by the square of the height in meters) above 25 are to be regarded as obese

Even mild obesity increases the risk of premature death and development of conditions such as diabetes, dyslipidemia, hypertension, atherosclerosis, gallbladder disease and certain types of cancer. In the industrialised western world the prevalence of obesity has increased significantly in the past few decades. Because of the high prevalence of obesity and its health consequences, its prevention and treatment should be a high public health priority.

Except for exercise, diet and food restriction, which is not feasible for a vast number of patients, no convincing treatments for reducing body weight effectively and acceptably currently exist. However, not only in view of the considerable problems directly related to obesity as described above, but also due to the important effect of obesity as a risk factor in serious (and even fatal) and common diseases, it is important to find pharmaceutical compounds which are useful in treatment and/or prevention of obesity.

When energy intake exceeds expenditure, the excess calories are stored predominately in adipose tissue, and if this net positive balance is prolonged, obesity results, i.e. there are two components to weight balance, and an abnormality on either side (intake or expenditure) can lead to obesity. This process may be counteracted by increasing energy expenditure (for instance via exercise) or decreasing energy intake (for instance by dieting). Only a few pharmacological treatments are available to date, e.g. treatment with Sibutramine™ (acting via serotonergic mechanisms; Abbott) or treatment with Orlistat™ (reducing fat uptake from the gut; Roche Pharm.), neither of which treatments reduces body weight effectively or to an acceptable degree. There is therefore a need for pharmaceutical compounds which may be useful in treatment and/or prevention of obesity, for instance by increasing energy expenditure or decreasing energy intake.

One way of increasing energy expenditure is to increase the metabolic rate. Oxidative phosphorylation in mitochondria, the energy from glucose metabolism and free fatty acids oxidation is used to drive the phosphorylation of ADP to ATP. When NADH and FADH₂ formed in the TCA cycle are oxidised back to NAD⁺ and FAD, respectively, protons are pumped out of the mitochondrial matrix. The resulting pH gradient (matrix pH˜8 and outside pH˜7) and potential (˜170 mV, inside negative) across the inner mitochondrial membrane constitute the electrochemical proton gradient. As the effect of a one-unit pH difference corresponds to a potential of 61.5 mV, the electrochemical proton gradient exerts a proton-motive force of roughly −230 mV, which is the driving force for mitochondrial ATP synthesis.

When ATP consumption thus increases, the cells respond by increasing ATP synthesis, and consequently the inward flux of protons, through ATP synthase, the enzyme responsible for ATP synthesis, and thereby the metabolic rate is increased. Chemical uncouplers are compounds which can transport protons across membranes, and when protons are transported across the inner mitochondrial membrane, ATP synthase is bypassed. At the (alkaline) matrix side the proton is released and the deprotonated uncoupler returns to the intermembrane space where it picks up another proton. The cycling of the uncoupler (or ATP synthesis) and the resulting proton transport leads to an increased outward pumping of protons through an increased oxidation of NADH and FADH₂ by the respiration chain. The NADH concentration in the matrix will consequently drop. Since NADH feed-back inhibits three steps in the TCA cycle (NADH is the main regulator of the TCA cycle), the flux through the TCA cycle will increase. Hence, the metabolic rate will increase.

Compounds, such as chemical uncouplers, which act by increasing the metabolic rate may thus be useful for treating obesity, but also for treating other conditions (diseases or disorders) such as atherosclerosis, hypertension, diabetes, especially type 2 diabetes [NIDDM (non-insulin dependent diabetes mellitus)], dyslipidemia, coronary heart disease, gallbladder disease, osteoarthritis and various types of cancer, such as endometrial, breast, prostate and colon cancers, and risk of premature death as well as other conditions that are improved by a reduction in mitochondrial potential.

Furthermore, chemical uncouplers may reduce the formation of mitochondrial reactive oxygen species (ROS) that are assumed to be involved in insulin resistance [Houstis et al., Nature 440 (2006) 944-948], in diabetic late complications [Brownlee, Diabetes 54 (2005) 1615-1625], in the aging process (De Grey et al, Eur J. Biochem 269, 1995 ff (2002)) and in damage of heart tissue as well as neuronal tissue. It is therefore also possible that conditions affected by ROS may be reversed or halted by intervention by administration of chemical uncouplers. Examples of such conditions include diabetic microvascular diseases in the retina, in the renal glomerulus and in peripheral nerve cells.

The best known chemical uncoupler is 2,4-dinitrophenol (DNP), which has been shown to increase energy expenditure in humans as well as animals. Side effects thereof at higher doses include increased perspiration, vasodilatation, skin rashes, cataracts, neuritis and even death. Two fatalities amongst the first 100.000 persons treated with DNP, and the fact that the lowest lethal dose was only twice the average dose which gave a desired 50% increase in basal metabolic rate, resulting in a very narrow safety window, combined with other factors led to the removal of DNP from the market. Since then, there appear to have been no attempts to develop or market uncouplers for the treatment of obesity.

Whilst DNP is the best known chemical uncoupler, many other compounds are known to induce uncoupling. These include DNP derivatives such as 4,6-dinitro-o-cresol (Victoria Yellow) and 2,4-dinitro-1-naphthol (Martius Yellow), as well as structurally unrelated compounds such as 2,6-di-t-butyl-4-(2′,2′-dicyanovinyl)phenol) (SF6847) (also known as 2-(3,5-di-tert-butyl-4-hydroxy-benzylidene)-malononitrile), carbonylcyanide m-chlorophenylhydrazone (CCCP) and carbonylcyanide p-trifluoromethoxy-phenylhydrazone (FCCP) [Miyoshi H. et al., Quantitative Relationship between Protenophoric and Uncoupling Activities of Analogs of SF6847 (2,6-di-t-butyl-4-(2′,2′-dicyanovinyl)phenol), Biochimica et Biophysica Acta 891, 293-299 (1987)].

Another class of chemical uncouplers is provided by the salicylanilides, of which S-13 is the most potent compound discovered so far [Terada H. et al., Structural Requirements of Salicylanilides for Uncoupling Activity in Mitochondria Quantitative Analysis of Structure-Uncoupling Relationships, Biochimica et Biophysica Acta 936, 504-512 (1988)].

WO 00/06143 (Texas Pharmaceuticals Inc.) relates to a method for inducing intracellular hyperthermia comprising a step of administering a mitochondrial uncoupling agent, such as 2,4-dinitrophenol.

U.S. Pat. No. 4,673,691 (Bachynsky) relates to the use of 2,4-dinitrophenol for treating obesity.

Various salicylic anilide derivatives have been disclosed in the literature. By way of example, U.S. Pat. No. 4,025,647 discloses compounds of the formula

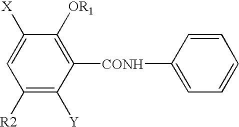

wherein R₁ may be hydrogen, X is secondary or tertiary alkyl, R₂ is alkanoyl, phenylsulfinyl, phenylsulfonyl, etc, and Y is hydrogen or methyl. The compounds have anthelmintic activity, especially against liver fluke.

EP 322823 discloses electrophotographic photoreceptors with the following formula

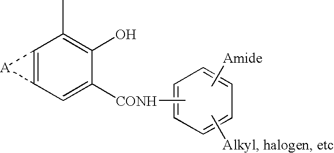

wherein A is a group of atoms necessary to condense the benzene ring with another ring.

WO 01/44172 discloses compounds of the formula

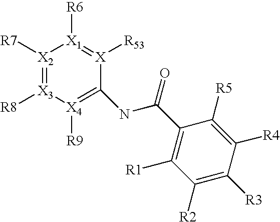

wherein all X's may be carbon, R1 may be hydroxy, and R2-R5 may be optionally substituted aryl, heteroaryl, alkylaryl, alkyl, ester, amide, etc. The compounds are inhibitors of serine proteases, urokinase, Factor Xa and Factor VIIa, and have utility as anticancer agents and as anticoagulants. R7 is amidine or guadinyl for all compounds specifically disclosed in this application.

WO 01/96944 discloses compounds of the formula

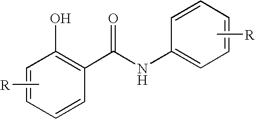

wherein R represents 0-4 substituents selected from alkyl, aryl, aralkyl, etc. The compounds are useful as components in colour photothermographic films. None of the specifically disclosed compounds have a branched alkyl or phenyl as substituent in the left-most phenyl ring.

WO 01/82924 discloses compounds of the formula

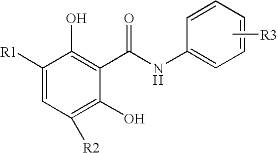

wherein R1-R3 represent hydrogen, alkyl, halogen, alkoxy, etc. The compounds are phosphate transport inhibitors.

### SUMMARY OF THE INVENTION

The present invention provides compounds of formula I, below:

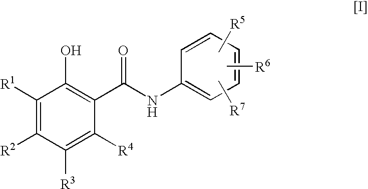

wherein
R¹ represents C₁₋₆alkyl, C₂₋₆alkenyl, C₂₋₆alkynyl, C₃₋₈cycloalkyl, C₄₋₈cycloalkenyl or aryl, all of which may optionally be further substituted with C₁₋₆alkyl, C₃₋₈cycloalkyl, C₄₋₈cycloalkenyl, or phenyl; or
R¹ represents a bicyclo-C₄₋₁₀alkyl or tricyclo-C₄₋₁₀alkyl;
and wherein, when R¹ is C₃₋₈cycloalkyl, bicyclo-C₄₋₁₀alkyl, tricyclo-C₄₁₀alkyl or aryl, R¹ may optionally be substituted with one or more substituents selected from halogen, hydroxy, cyano, nitro, C₁₋₆alkyl, C₂₋₆alkenyl, C₂₋₆alkynyl, C₃₋₈cycloalkyl, C₄₋₈cycloalkenyl, C₁₋₆alkoxy, C₁₋₆haloalkoxy and C₁₋₆haloalkyl; or
R¹ and R², or R² and R³, or R³ and R⁴, together with the benzene ring, form a bicyclic ring system which may optionally be substituted with one or more substituents selected from the group consisting of halogen, hydroxy, nitro, cyano, C₁₋₆alkyl, C₂₋₆alkenyl, C₂₋₆alkynyl, C₃₋₈cycloalkyl, C₄₋₈cycloalkenyl, C₁₋₆alkoxy, C₁₋₆haloalkoxy and C₁₋₆haloalkyl;
R² and R⁴ independently represent, hydrogen, halogen, C₁₋₆alkyl, C₂₋₆alkenyl, C₂₋₆alkynyl, C₃₋₈cycloalkyl, C₄₋₈cycloalkenyl or C₁₋₆alkoxy;
at least one of R⁵, R⁶ and R⁷ represents C₁₆haloalkoxy, and the remaining of R⁵, R⁶ and R⁷ independently represent hydrogen, nitro, cyano, halogen, C₁₋₆alkyl, C₂₋₆alkenyl, C₂₋₆alkynyl, C₃₋₈cycloalkyl, C₄₋₈cycloalkenyl, C₁₋₆haloalkyl, —OR¹⁰, —NR¹⁰R¹¹, —C(O)OR¹⁰, —COR¹⁰, —C(O)NR¹⁰R¹¹, —SH, —S(O)₂OR¹⁰, —S(O)₂NR¹⁰R¹¹, —S(O)ₙR¹¹, aryl or heteroaryl, wherein said aryl or heteroaryl may optionally be substituted with one or more C₁₋₆alkyl, halogen, hydroxy or phenyl;
R³ represents hydrogen, halogen, cyano, —OR¹⁰, —NR¹⁰R¹¹, —C(O)OR¹⁰, —COR¹⁰, —C(O)NR¹⁰R¹¹, —S(O)ₙR¹⁰, —S(O)₂NR¹⁰R¹¹, —NHCOR¹⁰, —NHSO₂R¹⁰, aryl or heteroaryl,
wherein said aryl or heteroaryl may optionally be substituted with one or more substituents selected from the group consisting of halogen, hydroxy, nitro, cyano, C₁₋₆alkyl, C₂₋₆alkenyl, C₂₋₆alkynyl, C₃₋₈cycloalkyl, C₄₋₈cycloalkenyl, C₁₋₆alkoxy, C₁₋₆ haloalkoxy and C₁₋₆haloalkyl;
n is 0, 1 or 2;
each R¹⁰ and R¹¹ are selected independently from the group consisting of hydrogen C₁₋₆alkyl, C₂₋₆alkenyl, C₂₋₆alkynyl, C₃₋₈cycloalkyl, C₄₋₈cycloalkenyl, C₁₋₆haloalkyl and C₁₋₆haloalkoxy;
and pharmaceutically acceptable salts, solvates and prodrugs thereof.

Further aspects of the invention include, but are not limited to:

a compound of the invention for use as a medicament (i.e. for use in therapy);

a pharmaceutical composition comprising one or more compounds of the invention;

a method for treating a disease or disorder which benefits from an increase in mitochondrial respiration, the method comprising administering to a patient in need thereof a therapeutically effective amount of a compound of the invention or a pharmaceutical composition of the invention, optionally in combination with one or more additional therapeutically active compounds as disclosed herein;

a method for: treating diseases or disorders as disclosed herein; for preventing weight gain in a subject; or for maintaining weight loss in a subject who has achieved a weight loss; the method comprising administering to a patient or subject in need thereof a therapeutically effective amount of a compound of the invention or a pharmaceutical composition of the invention, optionally in combination with one or more additional therapeutically active compounds as disclosed herein;

a method for increasing mitochondrial respiration in a subject, the method comprising administering a therapeutically effective amount of a compound of the invention or a pharmaceutical composition of the invention to the subject, optionally in combination with one or more additional therapeutically active compounds as disclosed herein;

a method for reducing the formation of reactive oxygen species in a subject, the method comprising administering a therapeutically effective amount of a compound of the invention or a pharmaceutical composition of the invention to the subject, optionally in combination with one or more additional therapeutically active compounds as disclosed herein;.

the use of a compound of the invention or a pharmaceutical composition of the invention in the manufacture of a medicament for the treatment of a disease or disorder which benefits from an increase in mitochondrial respiration; and

the use of a compound of the invention or a pharmaceutical composition of the invention in the manufacture of a medicament for the treatment of diseases or disorders as disclosed herein, for preventing weight gain in a subject, or for maintaining weight loss in a subject who has achieved a weight loss.

Definitions

In the present context, the term “alkyl” is intended to indicate a straight- or branched-chain, saturated monovalent hydrocarbon radical having from one to six carbon atoms, also denoted C₁₋₆alkyl. Typical C₁₋₆alkyl groups include, but are not limited to, e.g. methyl, ethyl, n-propyl, isopropyl, n-butyl, sec-butyl, isobutyl, tert-butyl, n-pentyl, 2-methylbutyl, 3-methylbutyl, 4-methylpentyl, 1,1-dimethylpropyl, 1,2-dimethylpropyl, 2,2-dimethylpropyl (neopentyl), n-hexyl, 1,2,2-trimethylpropyl, and the like. The term “C₁₋₆alkyl” as used herein also includes secondary C₃₋₆alkyl and tertiary C₄₋₆alkyl.

In the present context, the term “alkenyl” is intended to indicate a straight- or branched-chain monovalent hydrocarbon radical having from two to six carbon atoms and at least one carbon-carbon double bond. Typical C₂₋₆alkenyl groups include vinyl, allyl, 1-propenyl, 1,3 butadiene-1-yl, and the like.

In the present context, the term “alkynyl” is intended to indicate a straight- or branched-chain monovalent hydrocarbon radical having from two to six carbon atoms and at least one carbon-carbon triple bond, and optionally one or more carbon-carbon double bonds. Examples include ethynyl, propynyl and 3,4-pentadien-1-ynyl.

The terms “bicycloalkyl” and “tricycloalkyl” indicate fully saturated bicyclic and tricyclic structures, respectively. Examples include bicyclo[2.2.2]oct-1-yl, bicyclo[3.3.1]non-1-yl, 1-adamantyl and 2-adamantyl.

The term “halogen” is intended to indicate a substituent derived from an element in the seventh main group of the periodic system, which includes fluorine (giving rise to fluoro, F), chlorine (giving rise to chloro, Cl), bromine (giving rise to bromo, Br) and iodine (giving rise to iodo, I).

In the present context, the term “aryl” is intended to indicate a carbocyclic aromatic ring radical which may optionally be fused to another aromatic or non-aromatic ring. Typical aryl groups include phenyl, biphenylyl, indenyl, fluorenyl (1-fluorenyl , 2-fluorenyl, 3-fluorenyl or 4-fluorenyl), naphthyl (1-naphthyl or 2-naphthyl), anthracenyl (1-anthracenyl, 2-anthracenyl or 3-anthracenyl), 1,2,3,4-tetrahydro-quinolyl, 1,2,3,4-tetrahydro-naphthyl, and the like.

The term “heteroaryl”, as used herein, alone or in combination, refers to: an aromatic ring radical having, for instance, from 5 to 7 member atoms; or a fused aromatic ring system radical having, for instance, from 7 to 18 member atoms, and wherein at least one ring is aromatic;

and containing one or more heteroatoms selected from nitrogen, oxygen and sulfur; wherein N-oxides and sulfur monoxides and sulfur dioxides are permissible heteroaromatic substitutions. Examples include furanyl, thienyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl and indazolyl, thienyl(2-thienyl or 3-thienyl), furanyl(2-furanyl or 3-furanyl), indolyl, oxadiazolyl, isoxazolyl, thiadiazolyl, oxatriazolyl, thiatriazolyl, quinazolinyl, fluorenyl, xanthenyl, isoindanyl, benzhydryl, acridinyl, thiazolyl, pyrrolyl(1-pyrrolyl, 2-pyrrolyl or 3-pyrrolyl), pyrazolyl(1-pyrazolyl, 3-pyrazolyl, 4-pyrazolyl or 5-pyrazolyl), imidazolyl(1-imidazolyl, 2-imidazolyl, 4-imidazolyl or 5-imidazolyl), triazolyl(1,2,3-triazol-1-yl, 1,2,3-triazol-4-yl 1,2,3-triazol-5-yl, 1,2,4-triazol-3-yl or 1,2,4-triazol-5-yl), oxazolyl(2-oxazolyl, 4-oxazolyl or 5-oxazolyl), isoxazolyl(isoxazo-3-yl, isoxazo-4-yl or isoxaz-5-yl), isothiazolyl(isothiazo-3-yl, isothiazo-4-yl or isothiaz-5-yl)thiazolyl(2-thiazolyl, 4-thiazolyl or 5-thiazolyl), pyridinyl(2-pyridinyl, 3-pyridinyl or 4-pyridinyl), pyrimidinyl(2-pyrimidinyl, 4-pyrimidinyl, 5-pyrimidinyl or 6-pyrimidinyl), pyrazinyl, pyridazinyl(3-pyridazinyl, 4-pyridazinyl or 5-pyridazinyl), quinolinyl(2-quinolinyl, 3-quinolinyl, 4-quinolinyl, 5-quinolinyl, 6-quinolinyl, 7-quinolinyl or 8-quinolinyl), isoquinolinyl(1-isoquinolinyl, 3-isoquinolinyl, 4-isoquinolinyl, 5-isoquinolinyl, 6-isoquinolinyl, 7-isoquinolinyl or 8-isoquinolinyl), benzo[b]furanyl(2-benzo[b]furanyl, 3-benzo[b]furanyl, 4-benzo[b]furanyl, 5-benzo[b]furanyl, 6-benzo[b]furanyl or 7-benzo[b]furanyl), 2,3-dihydrobenzo[b]furanyl(2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl) or 7-(2,3-dihydro-benzo[b]furanyl)), benzo[b]thiophenyl(benzo[b]thiophen-2-yl, benzo[b]thiophen-3-yl, benzo[b]thiophen-4-yl, benzo[b]thiophen-5-yl, benzo[b]thiophen-6-yl or benzo[b]thiophen-7-yl), 2,3-dihydro-benzo[b]thiophenyl(2,3-dihydro-benzo[b]thiophen-2-yl, 2,3-dihydrobenzo[b]thiophen-3-yl, 2,3-dihydro-benzo[b]thiophen-4-yl, 2,3-dihydro-benzo[b]thiophen-5-yl, 2,3-dihydro-benzo[b]thiophen-6-yl or 2,3-dihydro-benzo[b]thiophen-7-yl), indolyl(1-indolyl, 2-indolyl, 3-indolyl, 4-indolyl, 5-indolyl, 6-indolyl or 7-indolyl), indazo(1-indazolyl, 3-indazolyl, 4-indazolyl, 5-indazolyl, 6-indazolyl or 7-indazolyl), benzimidazolyl(1-benzimidazolyl, 2-benzimidazolyl, 4-benzimidazolyl, 5-benzimidazolyl, 6-benzimidazolyl, 7-benzimidazolyl or 8-benzimidazolyl), benzoxazolyl(2-benzoxazolyl, 3-benzoxazolyl, 4-benzoxazolyl, 5-benzoxazolyl, 6-benzoxazolyl or 7-benzoxazolyl), benzothiazolyl(2-benzothiazolyl, 4-benzothiazolyl, 5-benzothiazolyl, 6-benzothiazolyl or 7-benzothiazolyl), carbazolyl(1-carbazolyl, 2-carbazolyl, 3-carbazolyl or 4-carbazolyl), 5H-dibenz[b,f]azepinyl(5H-dibenz[b,f]azepin-1-yl, 5H-dibenz[b,f]azepin-2-yl, 5H-dibenz[b,f]azepin-3-yl, 5H-dibenz[b,f]azepin-4-yl or 5H-dibenz[b,f]azepin-5-yl), 10,11-dihydro-5H-dibenz[b,f]azepinyl(10,11-dihydro-5H-dibenz[b,f]azepin-1-yl, 10,11-dihydro-5H-dibenz[b,f]azepin-2-yl, 10,11-dihydro-5H-dibenz[b,f]azepin-3-yl, 10,11-dihydro-5H-dibenz[b,f]azepin-4-yl or 10,11-dihydro-5H-dibenz[b,f]azepin-5-yl), benzo[1,3]dioxole (2-benzo[1,3]dioxole, 4-benzo[1,3]dioxole, 5-benzo[1,3]dioxole, 6-benzo[1,3]dioxole or 7-benzo[1,3]dioxole), and tetrazolyl (5-tetrazolyl or N-tetrazolyl).

In the present context the term “bicyclic ring system” as used herein, alone or in combination, is intended to indicate a carbocyclic or heterocyclic ring radical fused to another carbocyclic or heterocyclic ring radical, the two rings having two atoms in common. Typical bicyclic aromatic ring systems include, but are not limited to, naphthalene, quinoline, isoquinoline, indole and isoindole rings.

In the present context the term “cycloalkyl” is intended to indicate a cyclic saturated monovalent hydrocarbon radical having 3, 4, 5, 6, 7 or 8 ring carbon atoms. Examples hereof include cyclopropyl, cyclobutyl, cyclopentyl and cyclohexyl.

In the present context the term “cycloalkenyl” is intended to indicate a cyclic unsaturated monovalent hydrocarbon radical having 4, 5, 6, 7 or 8 ring carbon atoms. Examples hereof include cyclobutenyl, cyclopentenyl and cyclohexenyl.

In the present context, the term “alkoxy” is intended to indicate a radical of the formula —OR′, wherein R′ represents alkyl as indicated above.

The term “haloalkoxy” is intended to indicate an alkoxy as defined above substituted with one or more halogen substituents as defined above, e.g. fluoro, chloro, bromo or iodo. Examples include trihalomethoxy, such as trifluoromethoxy and trichloromethoxy, and 2,2,2-trichloro-1-ethoxy.

In the present context, the term “haloalkyl” is intended to indicate an alkyl as defined above substituted with one or more halogen substituents as defined above. Examples include trihalomethyl, such as trifluoromethyl and trichloromethyl; further examples include trihaloethyl, such as 2,2,2-trifluoro-1-ethyl and 2,2,2-trichloro-1-ethyl.

The term “nitro” designates the radical —NO₂.

The term “cyano” designates the radical —CN.

In the present context, the substituent designation S(O)ₙR^{x} refers to —SR^{x}, —SO—R^{x} or —SO₂R^{x};

As used herein, the term “solvate” refers to a complex of defined stoichiometry formed by a solute (in casu, a compound according to the present invention) and a solvent. Relevant solvents include, by way of example, water, ethanol and or acetic acid. When water is the solvent in question, a corresponding solvate is also referred to as a “hydrate”.

As used herein, the term “prodrug” includes derivatives of compounds of the invention such as biohydrolyzable amides and biohydrolyzable esters thereof, and also encompasses:
a) compounds in which the biohydrolyzable functionality in such a prodrug is encompassed in the compound according to the present invention; and
b) compounds which may be oxidized or reduced biologically at a given functional group to yield drug substances according to the present invention.
Examples of the latter type of functional group include 1,4-dihydropyridine, N-alkylcarbonyl-1,4-dihydropyridine, 1,4-cyclohexadiene, tert-butyl and the like.

In the present context, the term “pharmaceutically acceptable salt” is intended to indicate a salt which is not harmful to the patient. Such salts include pharmaceutically acceptable acid addition salts as well as pharmaceutically acceptable metal salts, ammonium salts and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acid, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic and p-toluenesulfonic acid, and the like. Further examples of pharmaceutically acceptable inorganic or organic acid addition salts include the pharmaceutically acceptable salts listed in J. Pharm. Sci. 1977, 66, 2, the contents of which are incorporated herein by reference. Examples of metal salts include lithium, sodium, potassium and magnesium salts, and the like. Examples of ammonium and alkylated ammonium salts include ammonium, methylammonium, dimethylammonium, trimethylammonium, ethylammonium, hydroxyethylammonium, diethylammonium, butylammonium and tetramethylammonium salts, and the like.

The term “therapeutically effective amount” of a compound as used herein refers to an amount sufficient to cure, alleviate or partially arrest the clinical manifestations of a given disease or disorder and its complications. An amount adequate to accomplish this is defined as a “therapeutically effective amount”. The amount that is effective for a particular therapeutic purpose will depend on the severity of the disease or injury as well as on the weight and general state of the subject. It will be understood that determination of an appropriate dosage may be achieved, using routine experimentation, by constructing a matrix of values and testing different points in the matrix, all of which is within the ordinary skills of a trained physician or veterinary.

The terms “treatment” and “treating” as used herein refer to the management and care of a patient for the purpose of combating a condition, disease or disorder. The term is intended to include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” is to be understood to refer to the management and care of a patient for the purpose of hindering the development of the condition, disease or disorder, and includes the administration of the active compounds to prevent the onset of symptoms or complications. The subject or patient to be treated is preferably a mammal, in particular a human being. Treatment of animals, such as dogs, cats, cows, sheep and pigs, is, however, also within the scope of the present invention.

### DESCRIPTION OF THE INVENTION

In one embodiment of a compound of the invention, R¹ and R², or R² and R³, or R³ and R⁴, together with the benzene ring, form a bicyclic aromatic ring system; in a further embodiment, R¹ and R², together with the benzene ring, form a bicyclic aromatic ringsystem.

In certain embodiments of compounds of the invention, the bicyclic aromatic ring system is a naphthalene ring.

In still further embodiments of a compound of the invention of the latter type, the compound has the formula II:

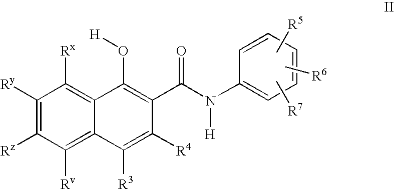

wherein
R^{x}, R^{y}, R^{z} and R^{v} independently are selected from the group consisting of halogen, hydroxy, cyano, nitro, C₁₋₆alkyl, C₂₋₆alkenyl, C₂₋₆alkynyl, C₃₋₈cycloalkyl, C₄₋₈cycloalkenyl, C₁₋₆alkoxy, C₁₋₆haloalkoxy and C₁₋₆haloalkyl;
and R³, R⁴, R⁵, R⁶ and R⁷ are as defined above for compounds of formula I.

In certain embodiments of compounds of compounds of the invention having the formula II, R^{y}, R^{z} and R^{v} are hydrogen; in other embodiments of compounds of the invention having the formula II, R^{y}, R^{z}, R^{v} and R⁴ are hydrogen; in still further embodiments of compounds of the invention having the formula II, R^{x} is methyl.

Specific, individual embodiments of compounds according to the present invention include each of the following compounds:
1-Hydroxy-naphthalene-2-carboxylic acid(4-cyano-2-trifluoromethoxy-phenyl)-amide;
2-Hydroxy-naphthalene-1-carboxylic acid(4-cyano-2-trifluoromethoxy-phenyl)-amide;
3-Hydroxy-naphthalene-2-carboxylic acid(4-cyano-2-trifluoromethoxy-phenyl)-amide;
1-Hydroxy-4-(4-methoxy-phenyl)-naphthalene-2-carboxylic acid(4-cyano-2-trifluoromethoxy-phenyl)-amide;
1-Hydroxy-4-p-tolyl-naphthalene-2-carboxylic acid(4-cyano-2-trifluoromethoxy-phenyl)-amide;
4-(4-Fluoro-phenyl)-1-hydroxy-naphthalene-2-carboxylic acid(4-cyano-2-trifluoromethoxy-phenyl)-amide;
1-Hydroxy-4-(3-trifluoromethyl-phenyl)-naphthalene-2-carboxylic acid (4-cyano-2-trifluoromethoxy-phenyl)-amide;
4-Bromo-1-hydroxy-naphthalene-2-carboxylic acid(4-cyano-2-trifluoromethoxy-phenyl)-amide;
1-Hydroxy-4-(4-trifluoromethylphenyl)naphthalene-2-carboxylic acid(4-cyano-2-trifluoromethoxyphenyl)amide;
1-Hydroxy-4-(3-methoxyphenyl)naphthalene-2-carboxylic acid(4-cyano-2-trifluoromethoxyphenyl)amide;
4-(3,4-Difluorophenyl)-1-hydroxynaphthalene-2-carboxylic acid(4-cyano-2-trifluoromethoxyphenyl)amide;
4-(3,5-Difluorophenyl)-1-hydroxynaphthalene-2-carboxylic acid(4-cyano-2-trifluoromethoxyphenyl)amide; and
4-(4-Cyanophenyl)-1-hydroxy-naphthalene-2-carboxylic acid(4-cyano-2-trifluoromethoxyphenyl)amide;
and pharmaceutically acceptable salts, solvates and prodrugs thereof.

Compounds according to formula I may comprise chiral carbon atoms, chiral sulfur atoms or carbon-carbon double bonds which may give rise to stereoisomeric forms, e.g. enantiomers, diastereomers and/or geometric isomers. The present invention relates to all such isomers, either in pure form or as mixtures thereof. Pure isomeric forms may be prepared from intermediates which are pure isomers themselves, by purification of a mixture of isomers after the synthesis, or by a combination of the two methods. Purification of isomeric forms is well known in the art, e.g. as described by Jaques in Enantiomers, Racemates and Resolution, Wiley, 1981.

Compounds of the present invention are, in general, useful in the treatment of conditions (diseases or disorders) that benefit from treatment with chemical uncouplers. Thus, for example, compounds of the present invention are useful in the treatment of conditions (diseases or disorders) that benefit from an increase in the mitochondrial respiration.

The compounds of the present invention are believed to be particularly well-suited for the treatment of obesity as such, or preventing weight gain, and for the treatment of conditions, diseases or disorders where obesity is involved in the etiology. In one aspect, the invention thus provides a method of treating the metabolic syndrome, insulin resistance, dyslipidemia, hypertension, obesity, type 2 diabetes, type 1 diabetes, diabetic late complications including cardiovascular diseases, cardiovascular disorders, disorders of lipid metabolism, neurodegenerative and psychiatric disorders, dysregulation of intraocular pressure including glaucoma, atherosclerosis, hypertension, coronary heart disease, gallbladder disease, osteoarthritis or cancer.

More specifically such conditions include the metabolic syndrome, type 2 diabetes (especially in obese patients), diabetes as a consequence of obesity, insulin resistance, hyperglycemia, prandial hyperglycemia, hyperinsulinemia, impaired glucose tolerance (IGT), impaired fasting glucose (IFG), increased hepatic glucose production, type 1 diabetes, LADA, pediatric diabetes, dyslipidemia (especially in obese patients), diabetic dyslipidemia, hyperlipidemia, hypertriglyceridemia, hyperlipoproteinemia, micro-/macroalbuminuria, nephropathy, retinopathy, neuropathy, diabetic ulcers, cardiovascular diseases, arteriosclerosis, coronary artery disease, cardiac hypertrophy, myocardial ischemia, heart insufficiency, congestional heart failure, stroke, myocardial infarction, arrythmia, decreased blood flow, erectile dysfunction (male or female), myopathy, loss of muscle tissue, muscle wasting, muscle catabolism, osteoporosis, decreased linear growth, neurodegenerative and psychiatric disorders, Alzheimers disease, neuronal death, impaired cognitive function, depression, anxiety, eating disorders, appetite regulation, migraine, epilepsia, addiction to chemical substances, disorders of intraocular pressure, bacterial infections, mycobacterial infections. In the present context cancer is intended to include forms such as hematological cancer, e.g. leukemia, acute myeloid leukemia, chronic myeloid leukemia, chronic lymphatic leukemia, myelodysplasia, multiple myeloma, Hodgkin's disease, or solid tumor forms, such as fibrosarcom, small or non-small cell long carcinoma, gastric, intestinal or colorectal cancer, prostate, endometrial, ovarian or breast cancer, brain, head or neck cancer, cancer in the urinary tract, such as kidney or bladder cancer, malignant melanoma, liver cancer, uterine and pancreatic cancer.

In another embodiment, the invention relates to the use of a chemical uncoupler compound according to the present invention for maintaining a weight loss.

The use of compounds according to the present invention in the treatment of obesity may very likely reduce or eliminate side-effects such as irritation of the skin, glaucoma, etc., that are known to occur in connection with treatment of obesity with DNP and other chemical uncouplers that have narrow safety windows.

Uncouplers may also reduce insulin release from β-cells, and may thus be useful in providing β-cell rest. Inducing β-cell rest may be useful in connection with β-cell transplantation, and it has also been described that inducing β-cell rest may be useful in preventing diabetes. Compounds of the invention are thus believed to be useful in the treatment of a patient for the purpose of providing β-cell rest.

Obesity drugs which regulate the appetite and reduce food intake often suffer from lack of long-term efficiency in terms of body weight loss because the body in response to the treatment lowers the rate of the metabolism. In contrast hereto, compounds of the present invention increase the metabolism, and they are therefore believed to be particularly suited for maintaining a weight loss.

Compounds of the present invention are also believed to be particularly well suited for the treatment of conditions, diseases or disorders where reactive oxygen species are involved in the etiology, and wherein a reduction in the amount of reactive oxygen species is beneficial. In one embodiment, the invention thus provides a method of treating, and in particular preventing, ageing and damage to the heart, endothelial cells and neuronal tissue, diabetic microvascular diseases in the retina, the renal glomerus and the peripheral nerve cells, the method comprising administering to a patient in need thereof a therapeutically effective amount of one or more compound of the present invention to a patient need thereof.

The subject (patient) may be any mammal suffering from a condition benefiting from increased mitochondrial respiration. Such mammals may include, for instance, horses, cows, sheep, pigs, mice, rats, dogs, cats, primates such as chimpanzees, gorillas and rhesus monkeys, and, in particular, humans.

It is well-known that many compounds used to combat insects or parasites, i.e. insecticides or parasiticides, respectively, are chemical uncouplers. It is thus believed that chemical uncoupler compounds according to the present invention may be useful as insecticides or parasiticides.

In methods of the present invention, a compound of the present invention may be administered alone, or it may be administered or in combination with one or more other therapeutically active compounds. When administered in combination with one or more other therapeutically active compounds, administration of the compound of the invention and the one or more other (additional) therapeutically active compounds, respectively, may take place either concomitantly or sequentially, and in any suitable ratios. Such additional therapeutically active compounds may, for example, be selected from antidiabetic agents, antihyperlipidemic agents, antiobesity agents, antihypertensive agents and agents for the treatment of complications resulting from, or associated with, diabetes.

Suitable antidiabetic agents include insulin, GLP-1 (glucagon like peptide-1) derivatives such as those disclosed in WO 98/08871 (Novo Nordisk A/S), the contents of which are incorporated herein by reference, as well as orally active hypoglycemic agents.

Suitable orally active hypoglycemic agents include imidazolines, sulfonylureas, biguanides, meglitinides, oxadiazolidinediones, thiazolidinediones, insulin sensitizers, α-glucosidase inhibitors, agents acting on the ATP-dependent potassium channel of the pancreatic β-cells, e.g. potassium channel openers such as those disclosed in WO 97/26265, WO 99/03861 and WO 00/37474 (Novo Nordisk A/S), the contents of all of which are incorporated herein by reference, potassium channel openers such as ormitiglinide, potassium channel blockers such as nateglinide or BTS-67582, glucagon antagonists such as those disclosed in WO 99/01423 and WO 00/39088 (Novo Nordisk A/S and Agouron Pharmaceuticals, Inc.), the contents of both of which are incorporated herein by reference, GLP-1 agonists such as those disclosed in WO 00/42026 (Novo Nordisk A/S and Agouron Pharmaceuticals, Inc.), the contents of which are incorporated herein by reference, DPP-IV (dipeptidyl peptidase-IV) inhibitors, PTPase (protein tyrosine phosphatase) inhibitors, glucokinase activators, such as those described in WO 02/08209 to Hoffmann La Roche, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antihyperlipidemic agents and antilipidemic agents, compounds lowering food intake, and PPAR (peroxisome proliferator-activated receptor) and RXR (retinoid X receptor) agonists such as ALRT-268, LG-1268 or LG-1069.

In one embodiment of a method of the invention, a compound of the present invention may be administered in combination with insulin or an insulin analogue.

In a further embodiment, a compound of the present invention may be administered in combination with a sulfonylurea, e.g. tolbutamide, chlorpropamide, tolazamide, glibenclamide, glipizide, glimepiride, glicazide or glyburide.

In another embodiment, a compound of the present invention may be administered in combination with a biguanide, e.g. metformin.

In yet another embodiment of a method of the present invention, a compound of the present invention may be administered in combination with a meglitianide, e.g. repaglinide or senaglinide/nateglinide.

In a still further embodiment, a compound of the present invention may be administered in combination with a thiazolidinedione insulin sensitizer, e.g. troglitazone, ciglitazone, pioglitazone, rosiglitazone, isaglitazone, darglitazone, englitazone, CS-011 /CI-1037 or T 174, or a compound disclosed in WO 97/41097 (e.g. 5-[[4-[3-methyl-4-oxo-3,4-dihydro-2-quinazolinyl]-methoxy]phenylmethyl]thiazolidine-2,4-dione), WO 97/41119, WO 97/41120, WO 00/41121 or WO 98/45292, the contents of all of which are incorporated herein by reference.

In another embodiment, a compound of the present may be administered in combination with an insulin sensitizer such as, e.g., GI 262570, YM-440, MCC-555, JTT-501, AR-H039242, KRP-297, GW-409544, CRE-16336, AR-H049020, LY510929, MBX-102, CLX-0940, GW-501516 or a compound disclosed in WO 99/19313 (NN622/DRF-2725), WO 00/50414, WO 00/63191, WO 00/63192, WO 00/63193 and WO 00/23425, WO 00/23415, WO 00/23451, WO 00/23445, WO 00/23417, WO 00/23416, WO 00/63153, WO 00/63196, WO 00/63209, WO 00/63190 or WO 00/63189, the contents of all of which are incorporated herein by reference.

In one embodiment, a compound of the present invention may be administered in combination with an α-glucosidase inhibitor, e.g. voglibose, emiglitate, miglitol or acarbose.

In a further embodiment, a compound of the present invention may be administered in combination with a glycogen phosphorylase inhibitor, e.g. a compound as described in WO 97/09040.

In another embodiment, a compound of the present invention may be administered in combination with a glucokinase activator.

In one embodiment, a compound of the present invention may be administered in combination with an agent acting on the ATP-dependent potassium channel of the pancreatic β-cells, e.g. tolbutamide, glibenclamide, glipizide, glicazide, BTS-67582 or repaglinide.

In another embodiment, a compound of the present invention may be administered in combination with nateglinide.

In one embodiment, a compound of the present invention may be administered in combination with an antihyperlipidemic agent or an antilipidemic agent, e.g. cholestyramine, colestipol, clofibrate, gemfibrozil, lovastatin, pravastatin, simvastatin, probucol or dextrothyroxine.

In another embodiment, a compound of the present invention may be administered in combination with more than one of the above-mentioned compounds, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In one embodiment, a compound of the present invention may be administered in combination with one or more antiobesity agents or appetite regulating agents. Such agents may be selected from the group consisting of CART (cocaine amphetamine regulated transcript) agonists, NPY (neuropeptide Y) antagonists, MC3 (melanocortin 3) agonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (fluoxetine, seroxat or citalopram), norepinephrine reuptake inhibitors (e.g. sibutramine), 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti related protein) inhibitors, H3 histamine antagonists such as those disclosed in WO 00/42023, WO 00/63208 and WO 00/64884, the contents of all of which are incorporated herein by reference, exendin-4, GLP-1 agonists and ciliary neurotrophic factor. Further agents of relevance are bupropion (antidepressant), topiramate (anticonvulsant), ecopipam (dopamine D1/D5 antagonist), naltrexone (opioid antagonist), peptide YY₃₋₃₆ (Batterham et al, Nature 418, 650-654 (2002)), and CB 1 endocannabinoid receptor antagonists, e.g. Acomplia™ (rimonabant).

In one embodiment, the antiobesity agent employed is leptin.

In another embodiment, the antiobesity agent employed is a lipase inhibitor, e.g. orlistat.

In a further embodiment, the antiobesity agent employed is an adrenergic CNS-stimulating agent, e.g. dexamphetamine, amphetamine, phentermine, mazindol, phendimetrazine, diethylpropion, fenfluramine or dexfenfluramine.

In another embodiment, a compound of the present invention may be administered in combination with one or more antihypertensive agents. Examples of relevant antihypertensive agents are: β-blockers such as alprenolol, atenolol, timolol, pindolol, propranolol and metoprolol; ACE (angiotensin converting enzyme) inhibitors such as benazepril, captopril, enalapril, fosinopril, lisinopril, quinapril and ramipril; calcium channel blockers such as nifedipine, felodipine, nicardipine, isradipine, nimodipine, diltiazem and verapamil; and a-blockers such as doxazosin, urapidil, prazosin and terazosin.

It should be understood that treatment of a subject in need thereof with any suitable combination of a compound according to the invention with diet and/or exercise and/or with one or more of the above-mentioned compounds, and optionally with one or more other active substances is considered to be within the scope of the present invention.

The present invention also provides pharmaceutical compositions comprising, as an active ingredient, at least one compound of the present invention, preferably in a therapeutically effective amount, suitable for use in any of the methods according to the present invention, together with one or more pharmaceutically acceptable carriers or excipients. Such pharmaceutical compositions may further comprise any of the further (additional) therapeutically active compounds as indicated above.

The pharmaceutical composition is preferably in unit dosage form, comprising from about 0.05 mg to about 1000 mg, preferably from about 0.1 mg to about 500 mg, and most preferably from about 0.5 mg to about 200 mg of a compound suitable for any of the methods described above.

Pharmaceutical Compositions

The compounds of the present invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 20^{th} Edition, Gennaro, Ed., Mack Publishing Co., Easton, Pa., 2000.

The pharmaceutical composition may be specifically formulated for administration by any suitable route, such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as hard or soft capsules, tablets, troches, dragees, pills, lozenges, powders and granules. Where appropriate, they can be prepared with coatings such as enteric coatings, or they can be formulated so as to provide controlled release of the active ingredient, such as sustained or prolonged release, according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions or emulsions, as well as sterile powders to be reconstituted in sterile injectable solutions or dispersions prior to use. Depot injectable formulations are also regarded as being within the scope of the present invention.

Other suitable administration forms include suppositories, sprays, ointments, cremes, gels, inhalants, dermal patches, implants, etc.

A typical oral dosage is in the range of from about 0.001 to about 100 mg/kg body weight per day, preferably from about 0.01 to about 50 mg/kg body weight per day, and more preferably from about 0.05 to about 10 mg/kg body weight per day, administered in one or more doses such as 1-3 doses. The exact dosage will depend upon the frequency and mode of administration, the sex, age, weight and general condition of the subject treated, the nature and severity of the condition treated and any concomitant diseases to be treated, and other factors evident to those skilled in the art.

The formulations may conveniently be prepared in unit dosage form by methods known to those skilled in the art. A typical unit dosage form for oral administration one or more times per day, such as 1-3 times per day, may contain from 0.05 to about 1000 mg, preferably from about 0.1 to about 500 mg, and more preferably from about 0.5 mg to about 200 mg of a compound of the invention.

For parenteral routes such as intravenous, intrathecal, intramuscular and similar administration, typical dosages are in the order of about half the dosage employed for oral administration.

A compound for use according to the present invention is generally utilized as the free substance or as a pharmaceutically acceptable salt thereof. Examples of the latter are: an acid addition salt of a compound having a free base functionality, and a base addition salt of a compound having a free acid functionality. The term “pharmaceutically acceptable salt” refers to a non-toxic salt of a compound for use according to the present invention, which salts are generally prepared by reacting a free base with a suitable organic or inorganic acid, or by reacting an acid with a suitable organic or inorganic base. When a compound for use according to the present invention contains a free base functionality, such salts are prepared in a conventional manner by treating a solution or suspension of the compound with a chemical equivalent of a pharmaceutically acceptable acid. When a compound for use according to the present invention contains a free acid functionality, such salts are prepared in a conventional manner by treating a solution or suspension of the compound with a chemical equivalent of a pharmaceutically acceptable base. Physiologically acceptable salts of a compound with a hydroxy group include the anionic form of the compound in combination with a suitable cation, such as sodium or ammonium ion. Other salts which are not pharmaceutically acceptable may be useful in the preparation of compounds of the invention, and these form a further aspect of the invention.

For parenteral administration, solutions of compounds for use according to the present invention in sterile aqueous solution, in aqueous propylene glycol or in sesame or peanut oil may be employed. Aqueous solutions should be suitably buffered where appropriate, and the liquid diluent rendered isotonic with, e.g., sufficient saline or glucose. Aqueous solutions are particularly suitable for intravenous, intramuscular, subcutaneous and intraperitoneal administration. The sterile aqueous media to be employed are all readily available by standard techniques known to those skilled in the art.

Suitable pharmaceutical carriers include inert solid diluents or fillers, sterile aqueous solutions and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax. The pharmaceutical compositions formed by combining the compounds for use according to the present invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units, such as capsules or tablets, which each contain a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

Compositions intended for oral use may be prepared according to any known method, and such compositions may contain one or more agents selected from the group consisting of sweetening agents, flavouring agents, colouring agents and preserving agents in order to provide pharmaceutically elegant and palatable preparations. Tablets may contain the active ingredient(s) in admixture with non-toxic pharmaceutically acceptable excipients which are suitable for the manufacture of tablets. These excipients may, for example, be: inert diluents, such as calcium carbonate, sodium carbonate, lactose, calcium phosphate or sodium phosphate; granulating and disintegrating agents, for example corn starch or alginic acid; binding agents, for example, starch, gelatine or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. No. 4,356,108, U.S. Pat. No. 4,166,452 and U.S. Pat. No. 4,265,874, the contents of which are incorporated herein by reference in their entirety, to form osmotic therapeutic tablets for controlled release.

Formulations for oral use may also be presented as hard gelatine capsules where the active ingredient is mixed with an inert solid diluent, for example, calcium carbonate, calcium phosphate or kaolin, or a soft gelatine capsules wherein the active ingredient is mixed with water or an oil medium, for example peanut oil, liquid paraffin, or olive oil.

Aqueous suspensions may contain the compound for use according to the present invention in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide such as lecithin, or condensation products of an alkylene oxide with fatty acids, for example polyoxyethylene stearate, or condensation products of ethylene oxide with long chain aliphatic alcohols, for example, heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as a liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example, sweetening, flavouring, and colouring agents may also be present.

The pharmaceutical compositions comprising compounds for use according to the present invention may also be in the form of oil-in-water emulsions. The oily phase may be a vegetable oil, for example, olive oil or arachis oil, or a mineral oil, for example a liquid paraffin, or a mixture thereof. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulated according to the known methods using suitable dispersing or wetting agents and suspending agents described above. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are conveniently employed as solvent or suspending medium. For this purpose, any bland fixed oil may be employed using synthetic mono- or diglycerides. In addition, fatty acids such as oleic acid find use in the preparation of injectables.

The compositions may also be in the form of suppositories for rectal administration of the compounds of the invention. These compositions can be prepared by mixing the drug with a suitable non-irritating excipient which is solid at ordinary temperatures but liquid at the rectal temperature and will thus melt in the rectum to release the drug. Such materials include, for example, cocoa butter and polyethylene glycols.

For topical use, creams, ointments, jellies, solutions of suspensions, etc., containing the compounds of the invention may be employed. In the context of the present invention, formulations for topical application include mouth washes and gargles.

Compounds of the present invention may also be administered in the form of liposome delivery systems, such as small unilamellar vesicles, large unilamellar vesicles, and multilamellar vesicles. Liposomes may be formed from a variety of phospholipids, such as cholesterol, stearylamine, or phosphatidylcholines.

In addition, some compounds of the present invention may form solvates with water or common organic solvents. Such solvates are also encompassed within the scope of the invention.

Thus, a further embodiment provides a pharmaceutical composition comprising a compound according to the present invention, or a pharmaceutically acceptable salt, solvate, or prodrug thereof, and one or more pharmaceutically acceptable carriers, excipients, or diluents.

If a solid carrier is used for oral administration, the preparation may be tabletted, placed in a hard gelatine capsule in powder or pellet form, or may be in the form of a troche or lozenge. The amount of solid carrier will vary widely, but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet that may be prepared by conventional tabletting techniques may contain:

| | | |
|---|---|---|
| Core: | | |
| Active compound (as free compound or salt thereof) | | 5.0 mg |
| Lactosum Ph. Eur. | | 67.8 mg |
| Cellulose, microcryst. (Avicel) | | 31.4 mg |
| Amberlite ® IRP88* | | 1.0 mg |
| Magnesii stearas Ph. Eur. | | q.s. |
| Coating: | | |
| Hydroxypropyl methylcellulose | approx. | 9 mg |
| Mywacett 9-40 T** | approx. | 0.9 mg |

*Polacrillin potassium NF, tablet disintegrant, Rohm and Haas.
**Acylated monoglyceride used as plasticizer for film coating.

If desired, the pharmaceutical composition comprising a compound according to the present invention may further comprise one or more additional therapeutically active substances, such as those described in the foregoing.

The present invention also provides methods for the preparation of compounds according to the present invention. The compounds can be prepared readily according to the following general procedures (in which all variables are as defined previously, above, unless otherwise indicated) using readily available starting materials, reagents and conventional synthesis procedures. In these reactions, it is also possible to make use of variants which are themselves known to those of ordinary skill in this art, but which are not mentioned in greater detail.

### EXAMPLES

HPLC-MS (Method A)

A Hewlett Packard series 1100 instrument is used. The HPLC pump is connected to two eluent reservoirs containing: (A) 0.01% TFA in water, (B) 0.01% TFA in acetonitrile. Gradient: 5%-100% acetonitrile linear during 7.5 min at 1.5 ml/min. The analysis is performed at 40° C. by injecting an appropriate volume of the sample (preferably 1 μl) onto the column which is eluted with a gradient of acetonitrile. Detection: 210 nm, analogue output from DAD (diode array detector), ELS (analogue output from ELS), and MS ionisation mode API-ES, Scan 100-1000 amu step 0.1 amu. After the DAD the flow is divided yielding approx 1 ml/min to the ELS and 0.5 ml/min to the MS.

HPLC-MS (Method B)

As Method A, except that the gradient is: 5%-100% acetonitrile linear during 4 min at 2.7 ml/min.

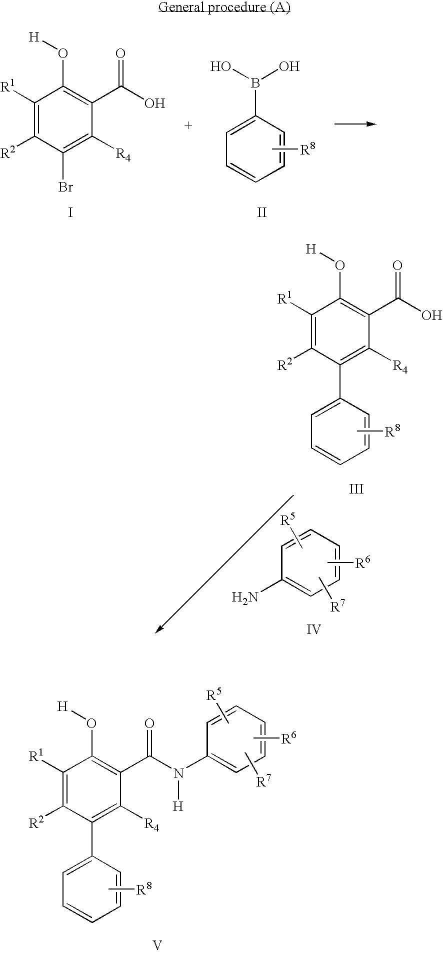

Step A:

To a solution of the bromo-substituted phenol I (1 equivalent) in water is added the appropriately substituted arylboronic acid II (1.1 equivalent). The appropriate palladium catalyst (palladium acetate 0.005 to 0.01 equivalents) is added together with the appropriate base (sodium carbonate, 3 equivalents). The reaction mixture is stirred at room temperature overnight or heated under reflux overnight. The reaction is followed by TLC or LC-MS. The reaction mixture is made acidic with a 1N hydrochloric acid solution, and the mixture is stirred at room temperature for 1-3 hours. The water phase is extracted with ethyl acetate. The organic phase is dried over sodium sulphate and evaporated. Pure compounds III are obtained by crystallisation from an organic solvent or by column chromatography.

The bromo-substituted phenols I are either commercially available or may be synthesized in one-step reactions from commercially available compounds by analogy with standard methods reported in the literature, e.g. Huang, Yunsheng et al.; J. Med. Chem. 2001, 44 (11), 1815-1826, or Mach, Robert H et al.; Med.Chem.Res. 1999, 9 (6) 355-373.

Step B:

Compound III (1 equivalent) is dissolved in xylene together with the appropriately substituted aniline IV (1-1.1 equivalent). The reaction mixture is heated to reflux and phosphorus trichloride, PCl₃ (0.33 equivalent) is carefully added. The reaction mixture is heated under reflux for 1-2 days. The reaction mixture is then allowed to cool to room temperature, and the product V is isolated by filtration and purified by recrystallisation from an organic solvent, or by column chromatography.

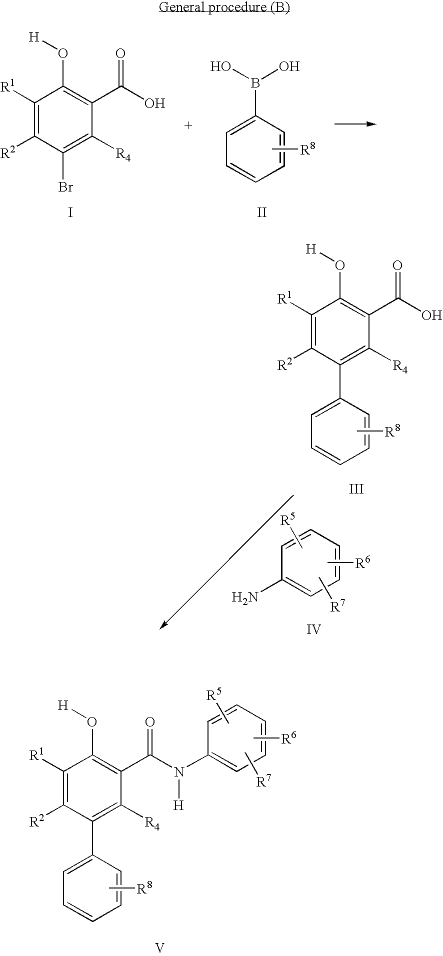

Step A:

The reaction is peformed in a manner analogous to general procedure A, step A.

Step B:

Compound III (1 equivalent) is dissolved in DMF, and 1,1′-carbonyldiimidazole (1.4 equivalent) is added under a nitrogen atmosphere. The mixture is stirred at room temperature for 2½ hour, and the appropriately substituted aniline IV (1-1.1 equivalent) is then added. The reaction mixture is heated under reflux for 24 hours, and the solvent is then evaporated. Acetonitrile and water are added to the crude residue, and the product V is isolated by filtration and purified by recrystallisation from an organic solvent, or by column chromatography.

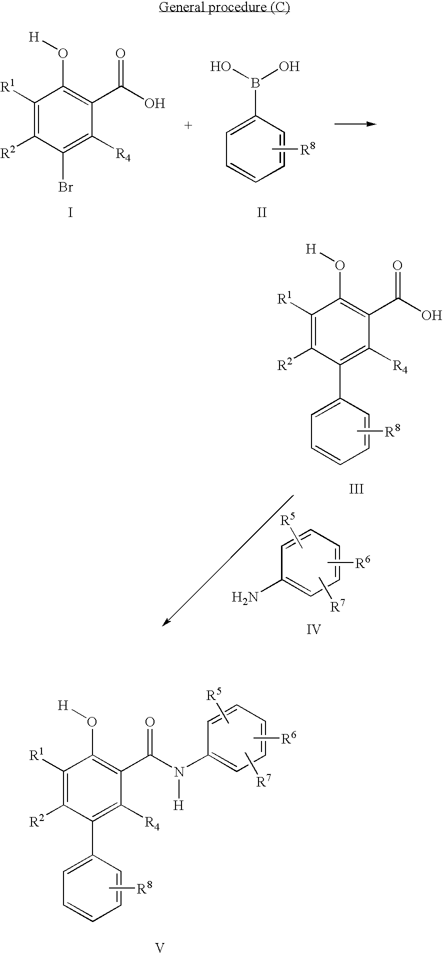

Step A:

The reaction is performed in a manner analogous to general procedure A, step A.

Step B:

Compound III (0.6 mmol) is dissolved in a mixture of thionylchloride (10 ml), acetonitrile (10 ml) and ethyl acetate (10 ml). The reaction mixture is then stirred for 1 hour at room temperature. The solvents are removed by evaporation, and the crude material is re-dissolved in a mixture of acetonitrile (10 ml) and ethyl acetate (20 ml). The appropriately substituted aniline IV (1-1.5 equivalent) is added. The reaction mixture is stirred under a nitrogen atmosphere at room temperature for 4 days. The solvent is removed by evaporation, and the crude compound is purified by column chromatography.

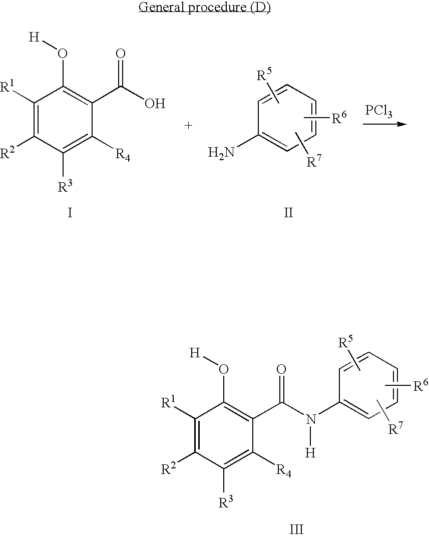

A mixture of compound I (1 equivalent) and the appropriately substituted aniline II (1.0-1.1 equivalent) in xylene is heated to reflux, and phosphorus trichloride (PCl₃) (0.33-1.0 equivalent) is carefully added. The reaction mixture is heated under reflux for 2-48 hours and allowed to cool to room temperature. The crude product is isolated by filtration or by evaporation to dryness, and pure compound III is obtained by recrystallization from an organic solvent, or by column chromatography.

### Example 1

### General Procedure (D)

### 1-Hydroxy-naphthalene-2-carboxylic acid(4-cyano-2-trifluoromethoxy-phenyl)-amide

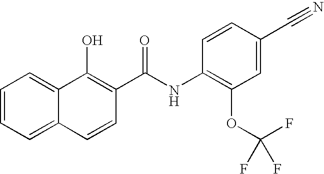

From 1-hydroxy-2-naphthoic acid and 4-amino-3-(trifluoromethoxy)benzonitrile; mp 178° C.; ¹H NMR (DMSO-d₆): δ 13.12 (s, 1H), 11.17 (s, 1H), 8.36 (d, J=8.08 Hz, 1H), 8.23 (d, J=8.59 Hz, 1H), 8.18-8.15 (m, 1H), 8.07 (d, J=8.59 Hz, 1H), 7.99 (dd, 1H), 7.95 (d, J=8.08 Hz, 1H), 7.73-7.67 (m, 1H), 7.65-7.59 (m, 1H), 7.54 (d, J=9.10 Hz, 1H); HPLC-MS (Method A): 373 (M+1), 395 (M+Na), Rₜ=4.86 min.

### Example 2

### General Procedure (D)

### 2-Hydroxy-naphthalene-1-carboxylic acid(4-cyano-2-trifluoromethoxy-phenyl)-amide

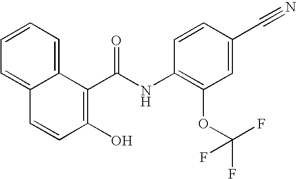

From 2-hydroxy-1-naphthoic acid and 4-amino-3-(trifluoromethoxy)benzonitrile; mp 169° C.; ¹H NMR (CD₃OD): δ 8.77-8.64 (br d, 1H), 8.27 (d, J=8.59, 1H), 7.88 (d, J=9.10, 1H), 7.82-7.74 (m, 3H), 7.52-7.45 (m, 1H), 7.38-7.31 (m, 1H), 7.20 (d, J=8.59, 1H); HPLC-MS (Method B): m/z=(4 min) m/z: 373 (M+H), 395 (M+Na) Rₜ=2.0 min.

### Example 3

### 3-Hydroxy-naphthalene-2-carboxylic acid(4-cyano-2-trifluoromethoxy-phenyl)-amide

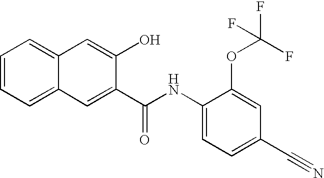

Step A:

### 3-Methoxy-naphthalene-2-carboxylic acid(4-cyano-2-trifluoromethoxy-phenyl)-amide

The title compound is prepared from 3-methoxy-naphthalene-2-carboxylic acid, 4-amino-3-(trifluoromethoxy)benzonitrile and PCl₃ in xylene according to the general procedure (D); ¹H NMR (CDCl₃): δ 10.90 (s, 1H), 9.01 (d, J=8.59 Hz, 1H), 8.88 (s, 1H), 7.95 (d, J=8.08 Hz, 1H), 7.80 (d, J=8.08 Hz, 1H), 7.67 (dd, J=8.59, 1.52 Hz, 1H), 7.62-7.56 (m, 2H), 7,48-7.43 (m, 1H), 7.33 (s, 1H), 4.19 (s, 3H); HPLC-MS (Method A): m/z: 387(M+1), 409 (M+Na), Rₜ=4.94 min.

Step B:

### 3-Hydroxy-naphthalene-2-carboxylic acid(4-cyano-2-trifluoromethoxy-phenyl)-amide

Boron tribromide (4.4 ml of a 0.1 M solution in dichloromethane) is added to a stirred solution of 3-methoxy-naphthalene-2-carboxylic acid(4-cyano-2-trifluoromethoxy-phenyl)-amide (0.17 g, 0.4 mmol) in dry dichloromethane (10 ml) at −70 ° C. under nitrogen. The dry-ice cooling bath is then removed, and the mixture is allowed to warm up to room temperature. After 30 minutes the mixture is extracted with saturated aqueous sodium bicarbonate (2×30 ml). The organic phase is dried over anhydrous MgSO₄, filtered and concentrated by evaporation to give the title compound. Yield 0.09 g (56%); white crystals, mp 248° C.; ¹H NMR (CDCl₃): δ 9.83 (s, 1H), 9.25 (s, 1H), 8.81 (d, J=8.59 Hz, 1H), 8.25 (s, 1H), 7.86 (d, J=8.08 Hz, 1H), 7.75-7.68 (m, 1H), 7.66-7.63 (m, 1 H), 7.59-7.53 (m, 1H), 7.43-7.36 (m, 2H); HPLC-MS (Method B): m/z: 373 (M+1), 395 (M+Na), Rₜ=2.19 min.

### Example 4

### General Procedure (A)

### 1-Hydroxy-4-(4-methoxy-phenyl)-naphthalene-2-carboxylic acid (4-cyano-2-trifluoromethoxyphenyl)-amide

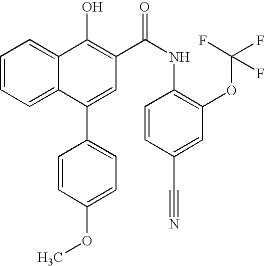

Step A:

From 4-bromo-1-hydroxy-naphthalene-2-carboxylic acid and 4-methoxy-phenylboronic acid. ¹H NMR (DMSO-d₆): δ 12.67 (brs, 1 H) 8.39 (d, J=8.08 Hz, 1 H) 7.79 (d, J=7.58 Hz, 1 H) 7.65 (m, 2 H) 7.61 (s, 1 H) 7.38 (d, J=8.59 Hz, 2 H) 7.08 (d, J=8.59 Hz, 2 H) 3.84 (s, 3 H); HPLC-MS (Method B): m/z=295 (M+1); Rₜ=1.82 min.

Step B:

From the product formed in step A and 4-cyano-2-trifluoromethoxy-phenyl aniline. ¹H NMR (DMSO-d₆): δ 13.25 (brs, 1 H) 11.22 (brs, 1 H) 8.44 (d, J=7.58 Hz, 1 H) 8.15 (m, 2 H) 7.98 (m, 2 H) 7.82 (d, J=7.58 Hz, 1 H) 7.66 (m, 2 H) 7.44 (d, J=8.59 Hz, 2 H) 7.12 (d, J=8.59 Hz, 2 H) 3.86 (s, 3 H); HPLC-MS (Method B): m/z=479 (M+1); Rₜ=2.68 min.

### Example 5

### General Procedure (A)

### 1-Hydroxy-4-p-tolyl-naphthalene-2-carboxylic acid(4-cyano-2-trifluoromethoxy-phenyl)-amide

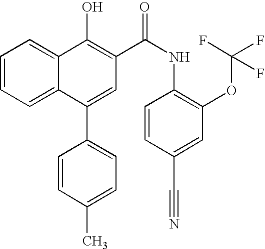

Step A:

From 4-bromo-1-hydroxy-naphthalene-2-carboxylic acid and 4-methyl-phenylboronic acid. ¹H NMR (DMSO-d₆): δ 12.65 (br s, 1 H) 8.39 (d, J=8.08 Hz, 1 H) 7.78 (d, J=8.08 Hz, 1 H) 7.65 (m, 2 H) 7.61 (s, 1 H) 7.33 (m, 4 H) 2.40 (s, 3 H); HPLC-MS (Method B): m/z=279 (M+1); Rₜ=1.96 min.

Step B:

From the product formed in step A and 4-cyano-2-trifluoromethoxy-aniline. ¹H NMR (DMSO-d₆): δ 13.28 (brs, 1 H) 11.26 (brs, 1 H) 8.43 (d, J=7.58 Hz, 1 H) 8.15 (m, 2 H) 7.98 (m, 2 H) 7.82 (d, J=7.58 Hz, 1 H) 7.66 (m, 2 H) 7.40 (d, J=8.08 Hz, 2 H) 7.36 (d, J=8.08 Hz, 2 H) 2.42 (s, 3 H); HPLC-MS (Method B): m/z=463 (M+1); Rₜ=2.78 min.

### Example 6

### General Procedure (A)

### 4-(4-Fluoro-phenyl)-1-hydroxy-naphthalene-2-carboxylic acid(4-cyano-2-trifluoromethoxyphenyl)-amide

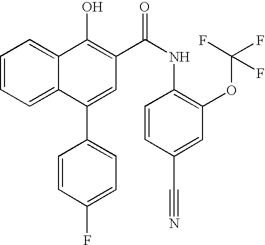

Step A:

From 4-bromo-1-hydroxy-naphthalene-2-carboxylic acid and 4-fluoro-phenylboronic acid. ¹H NMR (DMSO-d₆): δ 12.71 (br s, 1 H) 8.41 (d, J=7.58 Hz, 1 H) 7.70 (m, 3 H) 7.62 (s, 1 H) 7.50 (dd, J=8.59 and 5.56 Hz, 2 H) 7.35 (dd, J=9.1 Hz, 2 H); HPLC-MS (Method B): m/z=283 (M+1); Rₜ=1.85 min.

Step B:

From the product formed in step A and 4-cyano-2-trifluoromethoxy-phenyl aniline. ¹H NMR (DMSO-d₆): δ 13.31 (brs, 1 H) 11.25 (brs, 1 H) 8.45 (d, J=7.58 Hz, 1 H) 8.15 (m, 2 H) 8.02 (s, 1 H) 7.99 (dd, J=8.59 and 1.52 Hz, 1 H) 7.76 (d, J=7.58 Hz, 1 H) 7.69 (m, 2 H) 7.56 (m, 2 H) 7.40 (m, 2 H); HPLC-MS (Method B): m/z=467 (M+1); Rₜ=2.71 min.

### Example 7

### General Procedure (A)

### 1-Hydroxy-4-(3-trifluoromethyl-phenyl)-naphthalene-2-carboxylic acid(4-cyano-2-trifluoromethoxy-phenyl)-amide

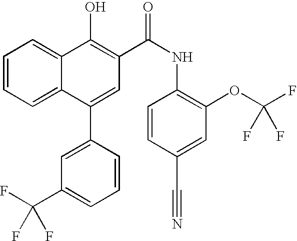

Step A:

From 4-bromo-1-hydroxy-naphthalene-2-carboxylic acid and 3-trifluoromethyl-phenylboronic acid.

Step B:

From the product formed in step A and 4-cyano-2-trifluoromethoxy-phenyl aniline. ¹H NMR (DMSO-d₆): δ 7.60-7.80 (m, 4 H), 7.81-7.90 (m, 3H), 7.99 (dd, J=8.48, 1.70 Hz, 1 H) 8.06 (s, 1 H) 8.11-8.20 (m, 2 H) 8.47 (d, J=7.16 Hz, 1 H) 11.23 (s, 1 H); HPLC-MS (Method B): m/z=517(M+1); Rₜ=2.87 min.

### Example 8

### General Procedure (D)

### 4-Bromo-1-hydroxy-naphthalene-2-carboxylic acid(4-cyano-2-trifluoromethoxy-phenyl)-amide

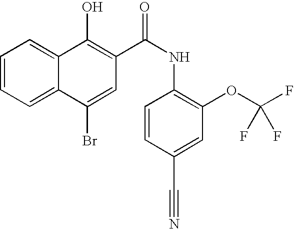

From 4-bromo-1-hydroxy-naphthalene-2-carboxylic acid and 4-cyano-2-trifluoromethoxy-aniline. ¹H NMR (DMSO-d₆): δ 7.73 (t, J=7.72 Hz, 1 H) 7.87 (t, J=7.72 Hz, 1 H) 7.99 (dd, J=8.48, 1.70 Hz, 1 H) 8.12 (d, J=8.29 Hz, 1 H) 8.16 (s, 1 H) 8.22 (d, J=8.67 Hz, 1 H) 8.38-8.47 (m, 2 H) 11.36 (s, 1 H); HPLC-MS (Method b): m/z=451, 453 (M+1); Rₜ=2.64 min.

### Example 9

### General Procedure (A)

### 1-Hydroxy-4-(4-trifluoromethylphenyl)naphthalene-2-carboxylic acid(4-cyano-2-trifluoromethoxyphenyl)amide

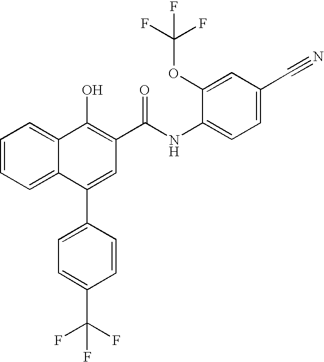

Step A:

From 4-bromo-1-hydroxy-naphthalene-2-carboxylic acid and 4-trifluoromethyl-phenylboronic acid. HPLC-MS (Method B): m/z=333 (M+1); Rₜ=2.54 min.

Step B:

From the product formed in step A and 4-cyano-2-trifluoromethoxyaniline. ¹H NMR (DMSO-d₆): δ 11.26 (brs, 1 H) 8.47 (d, J=7.16 Hz, 1 H) 8.17-8.13 (m, 2 H) 8.08 (s, 1 H) 7.99 (dd, J=8.29 and J=1.88 Hz, 1 H) 7.93 (d, J=8.29 Hz, 2 H) 7.80-7.65 (m, 5 H); HPLC-MS (Method B): m/z=517 (M+1); Rₜ=2.86 min.

### Example 10

### General Procedure (A)

### 1-Hydroxy-4-(3-methoxyphenyl)naphthalene-2-carboxylic acid(4-cyano-2-trifluoromethoxyphenyl)amide

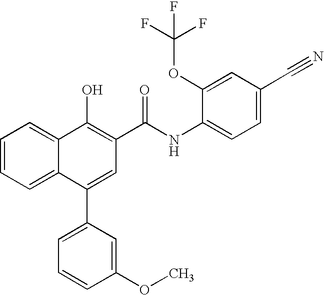

Step A:

From 4-bromo-1-hydroxy-naphthalene-2-carboxylic acid and 3-methoxy-phenylboronic acid. HPLC-MS (Method B): m/z=295 (M+1); Rₜ=2.15 min.

Step B:

From the product formed in step A and 4-cyano-2-trifluoromethoxyaniline. ¹H NMR (DMSO-d₆): δ 13.32 (brs, 1 H) 11.22 (brs, 1 H) 8.44 (d, J=7.54 Hz, 1 H) 8.16-8.13 (m, 2 H) 8.04 (s, 1 H) 7.98 (dd, J=8.29 and J=1.88 Hz, 1 H) 7.84 (d, J=7.54 Hz, 1 H) 7.72-7.63 (m, 2 H) 7.50-7.44 (m, 1 H) 7.09-7.05 (m, 3H) 3.83 (s, 3 H); HPLC-MS (Method B): m/z=479 (M+1); Rₜ=2.65 min.

### Example 11

### General Procedure (B)

### 4-(3,4-Difluorophenyl)-1-hydroxynaphthalene-2-carboxylic acid(4-cyano-2-trifluoromethoxyphenyl)amide

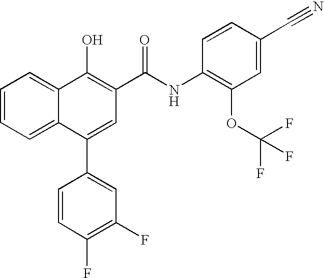

Step A:

From 4-bromo-1-hydroxy-naphthalene-2-carboxylic acid and 3,4-difluorophenylboronic acid. HPLC-MS (Method B): m/z=301 (M+1); Rₜ=2.20 min.

Step B:

From the product formed in step A and 4-cyano-2-trifluoromethoxyaniline. ¹H NMR (DMSO-d₆): δ 13.35 (brs, 1 H) 11.28 (brs, 1 H) 8.45 (d, J=8.08 Hz, 1 H) 8.17-8.13 (m, 2 H) 8.03 (s, 1 H) 7.99 (dd, J=8.59 and J=1.52 Hz, 1 H) 7.79 (d, J=7.58 Hz, 1 H) 7.73-7.59 (m, 4 H) 7.37 (m, 1 H); HPLC-MS (Method B): m/z=486 (M+2); Rₜ=2.71 min.

### Example 12

### General Procedure (A)

### 4-(3,5-Difluorophenyl)-1-hydroxynaphthalene-2-carboxylic acid(4-cyano-2-trifluoromethoxyphenyl)amide

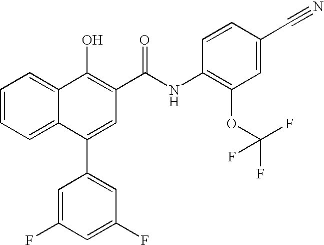

Step A:

From 4-bromo-1-hydroxy-naphthalene-2-carboxylic acid and 3,5-difluorophenylboronic acid. HPLC-MS (Method A): m/z=301 (M+1); Rₜ=4.48 min.

Step B:

From the product formed in step A and 4-cyano-2-trifluoromethoxyaniline. ¹H NMR (DMSO-d₆): δ 13.42 (brs, 1 H) 11.29 (brs, 1 H) 8.46 (d, J=8.08 Hz, 1 H) 8.17-8.13 (m, 2 H) 8.07 (s, 1 H) 7.99 (dd, J=8.59 and J=1.52 Hz, 1 H) 7.83 (d, J=7.58 Hz, 1 H) 7.74-7.66 (m, 2 H) 7.40-7.26 (m, 3 H); HPLC-MS (Method B): m/z=485 (M+1); Rₜ=2.79 min.

### Example 13

### General Procedure (C)

### 4-(4-Cyanophenyl)-1-hydroxy-naphthalene-2-carboxylic acid(4-cyano-2-trifluoromethoxyphenyl)amide

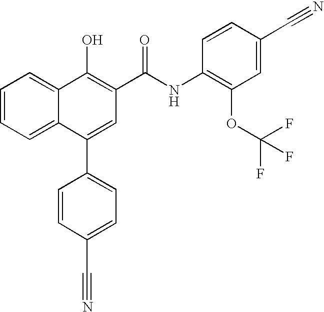

Step A:

4-(4-Cyanophenyl)-1-hydroxynaphthalene-2-carboxylic acid from 4-bromo-1-hydroxy-naphthalene-2-carboxylic acid and 4-cyanophenylboronic acid. HPLC-MS (Method B): m/z=290 (M+1); Rₜ=2.03 min.

Step B:

From the product formed in step A and 4-cyano-2-trifluoromethoxyaniline. ¹H NMR (DMSO-d₆): δ 13.40 (brs, 1 H) 11.28 (brs, 1 H) 8.47 (d, J=7.58 Hz, 1 H) 8.17-8.13 (m, 2 H) 8.07-8.03 (m, 3 H) 7.99 (dd, J=8.59 and J=1.52 Hz, 1 H) 7.78-7.66 (m, 5 H); HPLC-MS (Method B): m/z=474 (M+1); Rₜ=2.57 min.

Pharmacological Methods

Assay (I): Glucose Utilisation in a Human Epithelia Cell Line (FSK-4 Cells)

Assay Description:

The assay measures indirectly the activity of the respiratory chain in FSK-4 cells by using D-(6-³H(N))-glucose. The ³H-proton will first be released in the TCA cyclus and transported to the respiratory chain where it will be incorporated into water. The water is thereafter separated from the D-(6-³H(N))-glucose by evaporation. Finally, the radioactivity in the water is determined using a Topcounter.

Method:

FSK-4 cells obtained from ATCC (Maryland, USA), are cultured in growth medium (McCoy's medium with the following addition 100 units/ml penicillin and streptomycin and 10% FCS (fetal calf serum)) at 37° C. and 5% CO₂. All media are obtained from Gibco (Life Technologies, Maryland, USA) unless otherwise indicated.

At day zero the cells are harvested using trypsin-EDTA and washed in assay medium (MEM medium with the following addition 1× non-essential amino acids (M7145, 2 mM glutamin, 100 units/ml pencillin and streptomycin, 0.0075% sodium bicarbonate, 1 mM sodium pyrovate and 2% horse serum) using centrifugation. The cells are plated into single StripPlates wells (Corning B.V.Life Sciences, The Netherlands) that are placed into 24-well plates (Corning B.V. Life Sciences, The Netherlands) with a concentration of 1.5×10⁴ cells/100 μl assay medium/well. The cells are then incubated at 37° C. and 5% CO₂ overnight.

The next day the compounds to be tested are diluted to different concentrations in DMSO (Sigma, Missouri, USA) to 100 times final concentration. They are then diluted to a final concentration in assay medium containing 10 μCi/ml D-(6-³H(N))-glucose (PerkinElmer Life Sciences Inc., Boston, USA). The medium is removed from the cells and 200 μl of the compound dilutions are added in duplicates. The cells are then incubated for another 24 hours at 37° C. and 5% CO₂. Finally the cells are lysed by adding 50 μl 110% TCA (trichloroacetate). 300 μl of sterile water is then added to the 24-wells that surrounds the StripPlate wells. The plate is sealed with Top-seal-tape (Packard, PerkinElmer Life Sciences Inc., Boston, USA) and the plate is incubated in a heating cupboard at 50° C. to equilibrate the radioactive water formed in the respiratory chain into the water in the 24-well plate by evaporation. The plates are incubated for 8 hours, after which the heating cupboard is turned off. The top seal is removed when the samples have reached room temperature. One ml of scintillation liquid (Packard Microscient, PerkinElmer Life Sciences Inc., Boston, USA) is added to all the samples, and the radioactivity is determined using a Topcounter (Packard, PerkinElmer Life Sciences Inc., Boston, USA). Non-specific activity is determined by evaporating 200 μl of the dilution medium containing the D-(6-³H(N))-glucose into 300 μl sterile water, and total radioactivity is determined by counting 5 μl assay medium with 10 μCi/ml D-(6-³H(N))-glucose.

Calculations

The half-maximal concentration (EC₅₀) and maximal efficacy (Eₘₐₓ) are calculated by the Hill equation using GraphPad™ Prism 3.0 (GraphPad™ Software, Inc.). In studies where the linear slope is determined, the following concentration of the compound is used; 5×, 3×, 2×, 1.5×, 1.25×, 1×, 0.85×, 0.7×, 0.5×, 0.3×, 0.2× and 0× EC₅₀. From the percentage increase in glucose utilisation the linear slope is calculated using the Michaelis-Menten equation.

1. A compound according to formula I 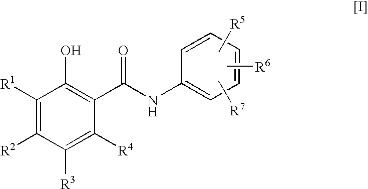
wherein
R¹ represents C₁₋₆alkyl, C₂₋₆alkenyl, C₂₋₆alkynyl, C₃₋₈cycloalkyl, C₄₋₈cycloalkenyl or aryl, all of which may optionally be further substituted with C₁₋₆alkyl, C₃₋₈cycloalkyl, C₄₋₈cycloalkenyl, or phenyl; or
R¹ represents a bicyclo-C₄₋₁₀alkyl or tricyclo-C₄₋₁₀alkyl;
and wherein, when R¹ is C₃₋₈cycloalkyl, bicyclo-C₄₋₁₀alkyl, tricyclo-C₄₋₁₀alkyl or aryl, R¹ may optionally be substituted with one or more substituents selected from halogen, hydroxy, cyano, nitro, C₁₋₆alkyl, C₂₋₆alkenyl, C₂₋₆alkynyl, C₃₋₈cycloalkyl, C₄₋₈cycloalkenyl, C₁₋₆alkoxy,
C₁₋₆haloalkoxy and C₁₋₆haloalkyl; or
R¹ and R², or R² and R³, or R³ and R⁴, together with the benzene ring, form a bicyclic ring system which may optionally be substituted with one or more substituents selected from the group consisting of halogen, hydroxy, nitro, cyano, C₁₋₆alkyl, C₂₋₆alkenyl, C₂₋₆alkynyl,
C₃₋₈cycloalkyl, C₄₋₈cycloalkenyl, C₁₋₆alkoxy, C₁₋₆haloalkoxy and C₁₋₆haloalkyl;
R² and R⁴ independently represent, hydrogen, halogen, C₁₋₆alkyl, C₂₋₆alkenyl, C₂₋₆alkynyl, C₃₋₈cycloalkyl, C₄₋₈cycloalkenyl or C₁₋₆alkoxy;
at least one of R⁵, R⁶ and R⁷ represents C₁₋₆haloalkoxy, and the remaining of R⁵, R⁶ and R⁷ independently represent hydrogen, nitro, cyano, halogen, C₁₋₆alkyl, C₂₋₆alkenyl, C₂₋₆alkynyl, C₃₋₈cycloalkyl, C₄₋₈cycloalkenyl, C₁₋₆haloalkyl, —OR¹⁰, —NR¹⁰R¹¹, —C(O)OR¹⁰, —COR¹⁰, —C(O)NR¹⁰R¹¹, —SH, —S(O)₂OR¹⁰, —S(O)₂NR¹⁰R¹¹, —S(O)ₙR¹¹, aryl or heteroaryl, wherein said aryl or heteroaryl may optionally be substituted with one or more C₁₋₆alkyl, halogen, hydroxy or phenyl;
R³ represents hydrogen, halogen, cyano, —OR¹⁰, —NR¹⁰R¹¹, —C(O)OR¹⁰, —COR¹⁰, —C(O)NR¹⁰R¹¹, —S(O)ₙR¹⁰, —S(O)₂NR¹⁰R¹¹, —NHCOR¹⁰, —NHSO₂R¹⁰, aryl or heteroaryl, wherein said aryl or heteroaryl may optionally be substituted with one or more substituents selected from the group consisting of halogen, hydroxy, nitro, cyano, C₁₋₆alkyl,
C₂₋₆alkenyl, C₂₋₆alkynyl, C₃₋₈cycloalkyl, C₄₋₈cycloalkenyl, C₁₋₆alkoxy, C₁₋₆ haloalkoxy and
C₁₋₆haloalkyl;
n is 0, 1 or 2;
each R¹⁰ and R¹¹ are selected independently from the group consisting of hydrogen C₁₋₆alkyl, C₂₋₆alkenyl, C₂₋₆alkynyl, C₃₋₈cycloalkyl, C₄₋₈cycloalkenyl, C₁₋₆haloalkyl and C₁₋₆haloalkoxy;
and pharmaceutically acceptable salts, solvates and prodrugs thereof.

2. The compound according to claim 1, wherein R¹ and R² or R² and R³ or R³ and R⁴, together with the benzene ring, form a bicyclic aromatic ring system.

3. The compound according to claim 2, wherein R¹ and R², together with the benzene ring, form a bicyclic aromatic ringsystem.

4. The compound according to claim 2, wherein said bicyclic aromatic ring system is a naphthalene ring.

5. The compound according to claim 3 having the formula II: 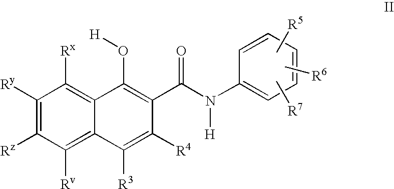
wherein
R^{x}, R^{y}, R^{z} and R^{v} independently are selected from the group consisting of halogen, hydroxy, cyano, nitro, C₁₋₆alkyl, C₂₋₆alkenyl, C₂₋₆alkynyl, C₃₋₈cycloalkyl, C₄₋₈cycloalkenyl,
C₁₋₆alkoxy, C₁₋₆haloalkoxy and C₁₋₆haloalkyl.

6. The compound according to claim 5, wherein R^{y}, R^{z} and R^{v} are hydrogen.

7. The compound according to claim 5, wherein R^{y}, R^{z}, R^{v} and R⁴ are hydrogen.

8. The compound according to claim 5 wherein R^{x} is methyl.

9. The compound according to claim 1, selected from the group consisting of:
1-Hydroxy-naphthalene-2-carboxylic acid(4-cyano-2-trifluoromethoxy-phenyl)-amide;
2-Hydroxy-naphthalene-1-carboxylic acid(4-cyano-2-trifluoromethoxy-phenyl)-amide;
3-Hydroxy-naphthalene-2-carboxylic acid(4-cyano-2-trifluoromethoxy-phenyl)-amide;
1-Hydroxy-4-(4-methoxy-phenyl)-naphthalene-2-carboxylic acid(4-cyano-2-trifluoromethoxy-phenyl)-amide;
1-Hydroxy-4-p-tolyl-naphthalene-2-carboxylic acid(4-cyano-2-trifluoromethoxy-phenyl)-amide;
4-(4-Fluoro-phenyl)-1-hydroxy-naphthalene-2-carboxylic acid(4-cyano-2-trifluoromethoxy-phenyl)-amide;
1-Hydroxy-4-(3-trifluoromethyl-phenyl)-naphthalene-2-carboxylic acid(4-cyano-2-trifluoromethoxy-phenyl)-amide;
4-Bromo-1-hydroxy-naphthalene-2-carboxylic acid(4-cyano-2-trifluoromethoxy-phenyl)-amide;
1-Hydroxy-4-(4-trifluoromethylphenyl)naphthalene-2-carboxylic acid(4-cyano-2-trifluoromethoxyphenyl)amide;
1-Hydroxy-4-(3-methoxyphenyl)naphthalene-2-carboxylic acid(4-cyano-2-trifluoromethoxyphenyl)amide;
4-(3,4-Difluorophenyl)-1-hydroxynaphthalene-2-carboxylic acid(4-cyano-2-trifluoromethoxyphenyl)amide;
4-(3,5-Difluorophenyl)-1-hydroxynaphthalene-2-carboxylic acid(4-cyano-2-trifluoromethoxyphenyl)amide; and
4-(4-Cyanophenyl)-1-hydroxy-naphthalene-2-carboxylic acid(4-cyano-2-trifluoromethoxyphenyl)amide;
and pharmaceutically acceptable salts, solvates and prodrugs thereof.

10. (canceled)

11. A pharmaceutical composition comprising one or more compounds according to claim 1.

12. A method for treating a disease or disorder benefiting from an increase in mitochondrial respiration, the method comprising administering to a patient in need thereof a therapeutically effective amount of a compound according to claim 1, optionally in combination with one or more additional therapeutically active compounds.

13. A method for: treating a disease or disorder selected among:
obesity per se; diseases or disorders where obesity is involved in the etiology; metabolic syndrome; insulin resistance; dyslipidemia; hypertension; type 2 diabetes; type 1 diabetes; diabetic late complications including cardiovascular diseases; disorders of lipid metabolism; neurodegenerative and psychiatric disorders; dysregulation of intraocular pressure including glaucoma; atherosclerosis; hypertension; coronary heart disease; gallbladder disease; osteoarthritis; and cancer;
preventing weight gain; or
maintaining weight loss
said method comprising administering to a patient in need thereof a therapeutically effective amount of a compound according to claim 1, optionally in combination with one or more additional therapeutically active compounds.

14. The method according to claim 13 for: treating a disease or disorder selected among obesity, atherosclerosis, hypertension, type 2 diabetes, dyslipidemia, coronary heart disease, osteoarthritis, gallbladder disease, endometrial cancer, breast cancer, prostate cancer, colon cancer, diabetic microvascular diseases in the retina, renal glomerulus and peripheral nerve cell apoptosis; preventing weight gain; or maintaining weight loss.

15. A method for increasing mitochondrial respiration in a subject in need thereof, said method comprising administering a therapeutically effective amount of a compound according to claim 1 to said subject, optionally in combination with one or more additional therapeutically active compounds.

16. A method for reducing the formation of reactive oxygen species in a subject in need thereof, said method comprising administering a therapeutically effective amount of a compound according to claim 1 to said subject, optionally in combination with one or more additional therapeutically active compounds.

17. The method according to claim 13, wherein a disease or disorder selected from atherosclerosis, hypertension, type 2 diabetes and dyslipidemia is treated, and wherein said patient is obese.

18. The method according to claim 13 for preventing weight gain or maintaining weight loss.

19. The method according to claim 13 for treating obesity.

20. The method according to claim 12, wherein administration of said one or more additional therapeutically active compounds is sequential or concomitant with administration of said compound.

21. The method according to claim 12, wherein said one or more additional therapeutically active compounds are selected from antidiabetic agents, antihyperlipidemic agents, antiobesity agents, antihypertensive agents and agents for the treatment of complications resulting from, or associated with, diabetes.

22. (canceled)

23. (canceled)

24. (canceled)

25. (canceled)
